(19) United States
(12) Patent Application Publication
(10) Pub. No.: US 2009/0104640 A1
(43) Pub. Date: Apr. 23, 2009

(54) Method of Evaluating Biological Material and Bioreactor Therefor

(75) Inventors: Valerie Barron, Galway (IE); Linda Ann Murphy, Dublin (IE); Edwin Lyons, Galway (IE); Abhay Pandit, Galway (IE); Peter Mc Hugh, Galway (IE)

Correspondence Address: FOLEY & LARDNER LLP, 975 PAGE MILL ROAD, PALO ALTO, CA, 94304, US

(73) Assignee: The National University of Ireland, Galway (IE)

(21) Appl. No.: 11/575,254

(22) PCT Filed: Sep. 14, 2005
(86) PCT No.: PCT/IE2005/000099

§ 371 Date: Dec. 10, 2007

(30) Foreign Application Priority Data
Sep. 16, 2004 (IE) S2004/0622

(51) Int. Cl. C12Q 1/02; C12M 1/00; G06F 19/00

(52) U.S. Cl. 435/29; 435/284.1; 702/19

(57) ABSTRACT

A method of evaluating biological material comprising forming a 3-dimensional scaffold of tubular biological material, delivering a fluid through the tubular biological material, and evaluating the biological material and in particular evaluating the effect of biomolecules, medical devices and medical devices containing biomolecules on biological material. The invention further relates to a bioreactor suitable for evaluating biological material.

### INTRODUCTION

The present invention relates to a method of evaluating biological material and in particular evaluating the effect of biomolecules, medical devices and medical devices containing biomolecules on biological material. The invention further relates to a bioreactor suitable for evaluating biological material.

In this specification the term “bioreactor” refers to a system for the creation, physical conditioning and testing of biological material. The term “biomolecule” refers to any molecule which can interact with living organisms and includes pharmaceutical drugs, new chemical entities, gene vectors and protein molecules. Evaluation of biological material to examine the effect of biomolecules and medical devices thereon is generally carried out by animal testing. Although valuable information can be obtained by animal testing, it is not without its drawbacks such as variation within species, limitations of the type of testing due to associated regulations and the cost of animal testing. Additionally, for ethical reasons, some people prefer not to carry out animal testing. There has therefore been a recent interest in tissue engineering and culturing biological material in vitro for subsequent testing.

PCT publication no. WO 96/34090 discloses an apparatus and method for sterilising, sealing, culturing, storing, shipping and testing vascular grafts. The vascular grafts are treated by placing a tube within the graft and expanding and contracting the tube using an alternating pressure source which applies a varying radial stress on the graft. The radial stress causes the cells to align themselves parallel to the axis of stress, therefore achieving the desired level of cell density in certain areas. The physical response of the vascular graft to the alternating pressure can be analysed using this apparatus.

US patent publication no. 2001/0031480 discloses a device and method for growing cells in an enclosed device. The device also includes a test chamber where the cells are removed to and where the efficacy of anti-cancer therapeutics can be tested on the cells. The disadvantage of testing cells however is that the results obtained only take into account the effect of that particular agent on those cells in isolation rather than when combined in the form of a tissue construct. Tissue comprises layers of cells interacting with each other, therefore the effect that a therapeutic would have on cells in isolation could be different to the effect that it would have on those cells when in the form of a tissue.

U.S. Pat. No. 6,096,550 discloses a method of testing a material comprising forming a biomembrane having at least some constituent matter of human or animal tissue on a polymer comprising a metal layer and testing the material on a biomembrane. Therefore the human or animal tissue is in a flat 2-dimensional form when being tested. The disadvantage of testing tissue in a 2-dimensional form however is that tissue exists in a 3-dimensional form in the human or animal body and therefore the effect that the test material will have on the 2-dimensional biomembrane will not directly correlate to the effect that it will have in tissue in the human or animal body.

Using the methods and systems of the prior art, it is therefore difficult to obtain meaningful and accurate results such as the effect a particular agent would have on a tissue when in the form that it exists in the human or animal body, i.e. the biological response of native tissue in vivo cannot be ascertained. Additionally it would not be possible to test the effect that the implantation of a medical device would have on tissue, by testing the component cells in isolation, as it would be impossible to predict the physical response of the tissue when those cells are combined in a tissue construct. Furthermore, using previously known systems it is difficult to evaluate tubular tissue such a cardiovascular tissue accurately.

There is therefore a need for a method of evaluating biological material in the form which it exists in the human or animal body.

### STATEMENTS OF INVENTION

According to the invention, there is provided a method of evaluating biological material comprising:
forming a 3-dimensional scaffold of tubular biological material;
delivering a fluid through the tubular biological material; and
evaluating the biological material;
characterised in that; the method further comprises the steps, not necessarily sequentially, of:
forming the 3-dimensional scaffold of tubular biological material so as to replicate native tissue in vivo;
transferring the biological material to an environment which simulates physiological conditions;
applying a test material to the biological material; and
analysing the interaction between the test material and the biological material.

The advantage of forming the 3-dimensional scaffold of tubular biological material so as to replicate native tissue in vivo and evaluating the biological material in this form is that more meaningful results can be obtained as to what would the effect that a particular test material would have on biological material in vivo.

Typically, the effect that a material would have on individual cells would be different to the effect that the material would have on those cells combined to form a tissue. Therefore by testing the tissue and more specifically a replica of native tissue rather than the individual cells the effect that a certain test material would have on the native tissue in vivo can be determined more accurately.

The advantage of evaluating the material under simulated physiological conditions is that all factors which would be present in vivo can be taken into consideration and a more accurate analysis of the interaction between the test material and the biological material can be achieved.

In one embodiment of the invention, the test material comprises biomolecules and the method further comprises:
labelling the biomolecules; and
delivering an amount of the labelled biomolecules to the biological material prior to transferring the biological material to the environment which simulates physiological conditions.

In another embodiment of the invention, the test material comprises biomolecules and the method further comprises:
labelling the biomolecules;
adding an amount of the labelled biomolecules to the fluid;
delivering the fluid and labelled biomolecules through the tubular biological material;
allowing the fluid and labelled biomolecules to interact with the biological material;
analysing the interaction between the labelled biomolecules and the biological material.

As the biomolecules are labelled, their interaction with the biological material can be clearly visualised. By applying the labelled biomolecules directly to the biological material it is easier to target specific areas of the material.

In a further embodiment of the invention, the test material comprises a medical device and the method further comprises:
implanting the medical device into the tubular biological material;
allowing the medical device and the biological material to interact;
analysing the interaction between the medical device and the biological material.

The advantage of using this method to test medical devices is that it is possible to test the mechanics and the physical properties of the device as well as the biological and physical response of the tissue simultaneously. Stents for example are generally constructed of either stainless steel or nitinol alloy, and it is therefore possible to also test the properties of the stent such as fatigue and corrosion properties.

In a still further embodiment of the invention, the method further comprises the steps of:
labelling biomolecules;
coating the medical device with an amount of the labelled biomolecules prior to implanting the medical device into the biological material; and
analysing the interaction between the labelled biomolecules, medical device and biological material.

The advantage of delivering biomolecules and implanting a medical device at the same time is that the biological response of the biological material to the biomolecule and the physical response of the biological material to the medical device can also be tested simultaneously. Therefore meaningful results for human tissue can be obtained. This is a useful method for testing for example the insertion of a stent covered with a certain drug, such as a drug which prevents the clogging of arteries (restenosis), into 3-dimensional tubular cardiovasular tissue. The physical properties of the stent can be tested as well as the biological effectiveness of the drug. It can also be seen whether applying a combination of stent and drug to the heart valve tissue has any adverse effect on the effectiveness of either the stent, the drug or both and/or the viability of the heart valve tissue. Additionally, it is more accurate to deliver biomolecules via a medical device.

Ideally, the medical device is selected from a group consisting of one or more of stent, artificial heart valve, cardiac patch and vascular graft.

Preferably, the fluid is delivered at a rate of between 1 and 5 l/min. The advantage of delivering the fluid at this rate is that this range is within physiological flow rate parameters.

Ideally, the fluid is selected from a group consisting of one or more of physiological saline, aldehyde solution, isotonic saline solution, albumin solution or suspension, tissue culture medium and blood. The advantage of using these types of fluid to deliver the labelled biomolecules is that they are physiologically compatible with and therefore will not have any adverse effect on the biological material.

Preferably, the biomolecules are labelled using one or more of magnetic labelling, radiolabelling, fluorescent labelling and thermal imaging. The advantage of labelling the biomolecules is that their distribution to and interaction with the biological material can be easily monitored. Thermal imaging can also be used to determine the viability of the cells within the tissue. Preferably fluorescent labelling or thermal imaging is used as these methods are safer and easier to use.

Ideally, the biological material is analysed using an instrument selected from the group consisting of one or more of probe, camera, sensor, laser and pressure transducer.

Preferably, the biological material is cardiovascular tissue selected from a group consisting of one or more of vascular graft tissue, heart valve tissue, artery tissue and cardiac muscle tissue.

In a further embodiment of the invention, the method further comprises:
prior to applying the test material to the biological material, carrying out the further step of:
determining the material, physical, and/or biological properties of the biological material.

The advantage of determining the material, physical, and/or biological properties of the biological material prior to applying the test material is that any changes due to biological material insertion can be examined. These properties can be determined by either visual methods or by using instruments in combination with analytical formulae and computer based calculation methods.

Preferably, the biological material is transferred to a bioreactor which simulates physiological conditions; and
the biological material is evaluated as a 3-dimensional scaffold under simulated physiological conditions within the bioreactor.

The invention further provides a bioreactor suitable for evaluating biological material. The bioreactor is kept in an incubator so that the conditions within the bioreactor can be more easily and accurately controlled to mimic physiological conditions within the human or animal body.

The invention also provides a computer program comprising program instructions for causing a computer to control the step of delivering the fluid through the tubular biological material. The advantage of using a computer program to control the step of delivering the fluid through the tubular biological material is that as the flow of the fluid through the scaffold is computer controlled, a physiological waveform is generated to pump the fluid through the scaffold, i.e. replicating blood flow in the body. In typical pulsatile systems a motor driven pump just pushes the fluid through the system, however the pattern of flow or waveform is not physiological.

The invention still further provides a computer program comprising program instructions for causing a computer to carry out the step of analysing the interaction between the test material and the biological material. By using a computer program to analyse this interaction this allows for the tissue to be constantly monitored and any change in the materials properties to be noted over a period of time. Additionally using analytical formulae and computer based calculation methods, certain properties of the biological material can be easily determined.

In one embodiment of the invention the computer program is embodied on a record medium.

In another embodiment of the invention the computer program is stored in a computer memory.

In a further embodiment of the invention the computer program is embodied in a read only memory

In a still further embodiment of the invention the computer program is carried in an electrical signal carrier.

### DETAILED DESCRIPTION OF THE INVENTION

The invention will be more clearly understood from the following description of some embodiments thereof given by way of example only with reference to the accompanying drawings in which:

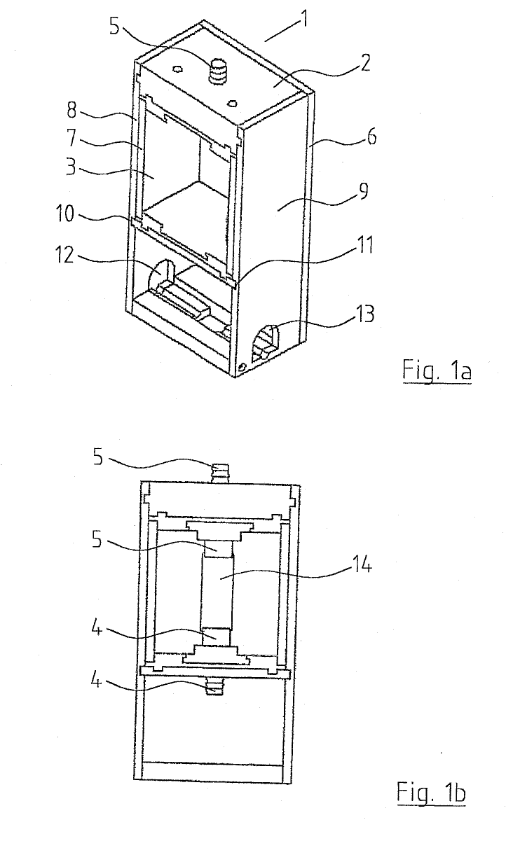

FIG. 1(a) is a perspective view of a bioreactor according to the invention.

FIG. 1(b) is a front view of the bioreactor illustrated in FIG. 1(a).

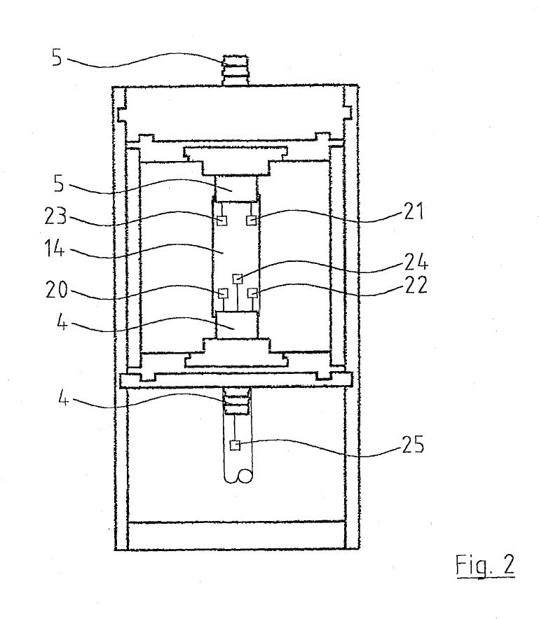

FIG. 2 is a front view of the bioreactor illustrated in FIGS. 1(a) and (b) showing the positioning of testing equipment.

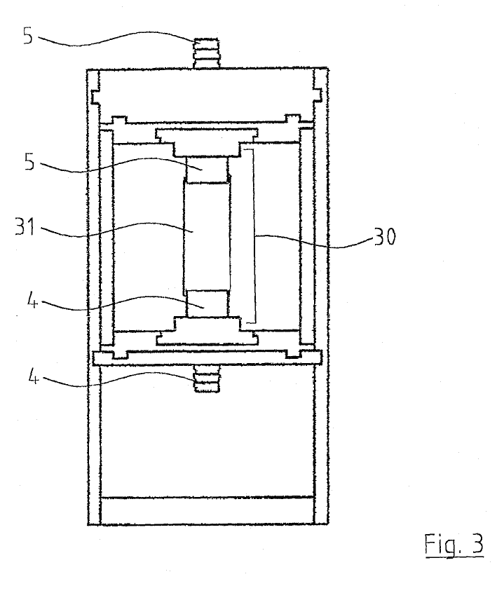

FIG. 3 is a front view of one construction of the bioreactor with a heart valve module.

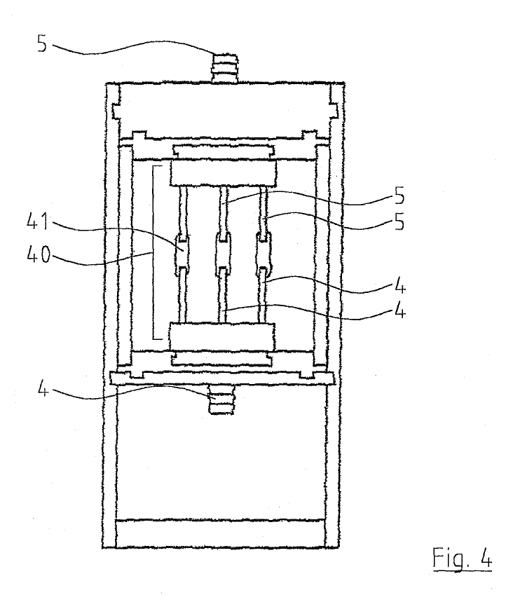

FIG. 4 is an alternative construction of the bioreactor with a vascular graft module.

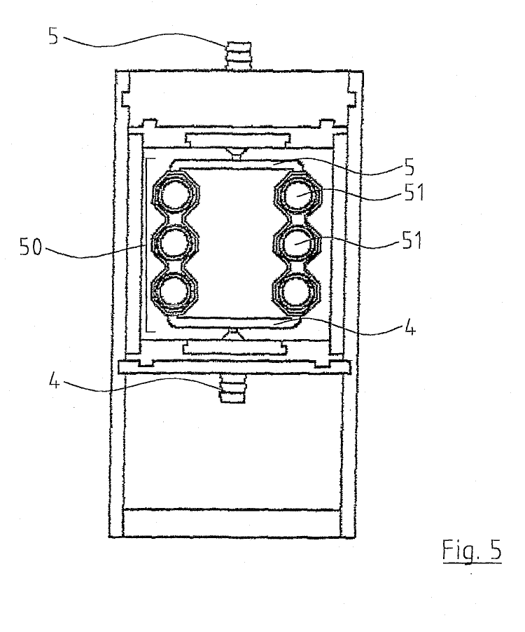

FIG. 5 is a further construction of the bioreactor with a gel module.

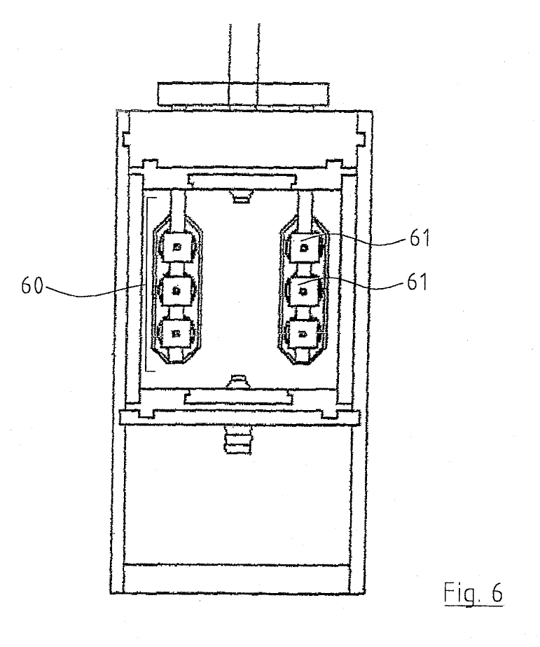

FIG. 6 is a further alternative construction of the bioreactor with a point bending module.

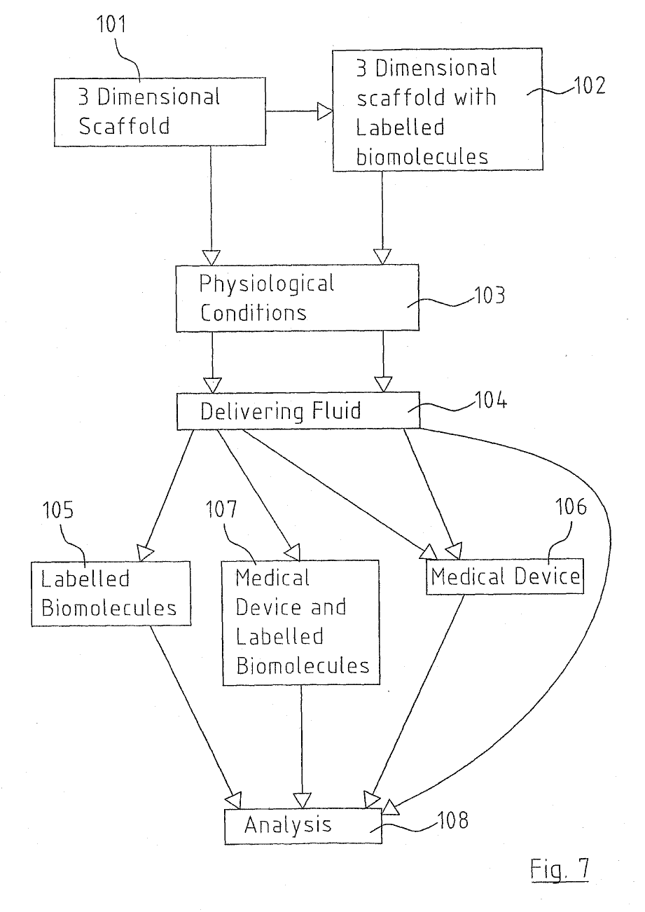

FIG. 7 is a process outline of a method of evaluating biological material according to the invention.

According to FIG. 1(a) there is provided a bioreactor indicated generally by reference numeral 1 comprising a housing (2), a tissue testing chamber (3), a fluid inlet pipe (4) and a fluid outlet pipe (5). The tissue testing chamber (3) can be released from the housing (2). The housing (2) comprises a back panel (6), a front panel (7) and a pair of side panels (8, 9).

When in situ in the housing (2) the tissue testing chamber (3) is held by grooves (10, 11) in the respective side panels (8, 9) which allow the tissue testing chamber (3) to be removed and replaced easily. Each side panel (8, 9) further comprises a pair of cutaway portions (12, 13) respectively through one of which a pipe is inserted which connects the fluid inlet pipe (4) to a reservoir (not shown).

The housing (2) can be constructed of any rigid material such as glass, plexi glass or any other suitable biocompatible material. The housing material can be either transparent or a viewing port can be provided in at least one wall of the housing (2) so that visual monitoring of the biological material within the tissue testing chamber (3) is permitted.

The tissue testing chamber (3) can be constructed of any material suitable for undergoing sterilisation and should be non-cytotoxic to the specific tissue being tested. Suitable materials include polyethylene terephthalate (PET), polyvinyl chloride (PVC), Teflon®, polycarbonate, stainless steel, polyethylene, acrylates such as polymethyl methacrylate, polymethyl acrylate, vinyl chloride-vinylidene chloride copolymers, polypropylene, urea, formaldehyde copolymer, melamine formaldehyde copolymer, polystyrene, polyamide, polytetrafluoroethylene, polyfluoratrichloroethylene, polyesters, phenol formaldehyde resins, polyvinyl butyryl, cellulose acetate, cellulose acetate propionate, ethylcellulose, polyoxymethylene and polyacrylonitryl. The material of construction should be a non-thrombogenic material so as not to promote clotting of the blood.

Sterilisation of the tissue testing chamber (3) may be in the form of chemical sterilisation such as treatment with ethylene oxide, acetylene oxide or peracetic acid, radiation such as with electron beam or gamma rays or by heat sterilisation with steam in an autoclave.

The panels of the tissue testing chamber (3) can be bonded together by means of a sealant such as silicone glue or mechanically screwed together in order to provide an air-tight seal. It will be appreciated that any sealant suitable for being sterilised and which is biocompatible for cardiovascular applications can be used.

Referring now to FIG. 1(b) a scaffold (14) connects the fluid inlet pipe (4) to the fluid outlet pipe (5). The term “scaffold” may refer to a construct of self-supporting biological material or to a construct of biological material surrounding and supported by a matrix of biocompatible material. Biocompatible materials such as collagen, expanded polytetrafluoroethylene (ePTFE), bioresorbable polymers such as (PGA/P4HB), PGA, and polyethyleneterephthalate (DACRON®) are suitable. The biocompatible material should either be porous, degradable or both. This is to ensure that when the fluid passes through the scaffold that it can access the biological material. The biological material may be any tissue engineered construct, a naturally formed biological construct, or decellurised material which replicates native tissue in vivo.

The bioreactor is stored in an incubator during use, to control conditions within the bioreactor to mimic physiological conditions. The CO₂ content is controlled within the incubator so that the CO₂ content within the bioreactor is in the region of 5%. There is an O₂ sensor within the bioreactor, to ensure that the O₂ levels within the bioreactor are in the region of 95%. Generally, this sensor is placed in the fluid outlet pipe (5) to monitor O₂ levels in the fluid exiting the tissue testing chamber (3) in the bioreactor (1). As soon as the O₂ levels fall below the required value, fresh media having sufficient O₂ is introduced into the bioreactor (1) via the fluid inlet pipe (4) to replenish depleted O₂ levels. The fresh media should generally have an oxygen content in the region of between 80 to 81 mg O₂/l at atmospheric pressure and ambient temperature.

The temperature within the bioreactor is also controlled by the incubator and should be in the region of 37° C. The pH levels are also monitored by testing the fluid exiting the bioreactor and should be in the region of 7. An increase or decrease in pH can also be counteracted by the introduction of fresh media.

In use, fluid enters the bioreactor (1) through the fluid inlet pipe (4) is delivered through the scaffold (14) and exits the bioreactor via the fluid outlet pipe (5). The fluid can be delivered in a pulsatile manner. Generally the pulsatile flow is at a rate of 60 beats/min however the pulsatile flow can be altered, to simulate flow for different blood pressures, i.e. simulate conditions in the heart for people with high blood pressure, people with low blood pressure, etc. Many commercially available pumps are suitable for providing pulsatile flow such as a peristaltic, piston or diaphragm pump. A linear actuator connected to stepper motors could also be used. The fluid can be any biocompatible fluid such as physiological saline, aldehyde solution, isotonic saline solution, albumin solution or suspension, tissue culture medium or blood. The fluid can furthermore comprise nutrients such as growth factors or other components such as serum or antibiotics.

The fluid flow is controlled in terms of composition, flow rate, pressure and temperature to provide biochemical and mechanical stimulation. The controls may be in the form of flow metres, pressure transducers, probes and thermometers attached to the scaffold (14) or fluid inlet or outlet pipes (4, 5).

It will be appreciated that culturing of the vascular graft tissue, heart valve tissue and other biological material can be carried out by culturing techniques which are well known by persons skilled in the art and utilising well known bioreactors. A vascular graft for example comprises three layers, namely the intima, i.e. the inner layer that consists of an endothelial cell lining and is closest to the blood flow, the media, the middle layer which consists of smooth muscle cells surrounded by collagen and elastin and the adventitia, the outer layer that consists of extra cellular matrix with fibroblasts, blood vessels and nerves. Culturing of a vascular graft therefore comprises seeding cells from each of these layers. Initially smooth muscle cells are grown on a scaffold material, either a degrading scaffold or a non-degrading scaffold, and stored in media to allow tissue growth to occur. The smooth muscle cell layer may be transferred to a bioreactor to enhance growth. Once these smooth muscle cells form a tissue layer, a fibroblast layer representing the adventitial layer cells are grown on top of the smooth muscle cell layer. Finally endothelial cells are seeded on the lumen side of the smooth muscle cell fibroblast sandwich. Growth factors may be used to enhance this endothelialisation.

Once the tissue has sufficient mechanical integrity it is transferred aseptically to the tissue testing chamber of the bioreactor for testing. Each type of biological material must satisfy certain conditions, in order to allow accurate results to be obtained. For example, in terms of mechanical properties, a vascular graft is required to withstand a normal physiological pressure in the 90-120 mm Hg range, have burst strength of the order of 1680 mm Hg, and suture retention strength of the order of 273 g. The vascular graft should also be of uniform thickness. Optimal vascular grafts will have a confluent endothelium and differentiated smooth muscle cells, collagen and elastin content, mechanical integrity and elastic moduli for suture retention and will be capable of withstanding arterial pressures. Furthermore, thickness, length, cell density across the thickness etc, should be as similar to a natural vessel as possible. In general the diameter of each vascular graft is in the region of 5 mm, but they can be engineered to thickness and length requirements.

With respect to heart valves such as mitral valves and tricuspid valves, the valve replacements should comprise epithelial tissue to form an endocardium and connective tissue.

Referring to FIG. 2, evaluation of the biological material may be carried out using a number of instruments. A camera (20) could be inserted into the scaffold (14) through the fluid inlet pipe (4).

A laser (21) could also be inserted through the fluid outlet pipe (5).

Pressure transducers (22, 23) could be placed at the fluid inlet and outlet pipes (4, 5) respectively.

A probe (24) such as a fluorescence probe could be inserted into the scaffold (14) via the fluid inlet pipe (4).

Sensors (25) such as a flow rate sensor can also be positioned in either the fluid inlet or outlet pipes (4, 5).

It will be appreciated that each of the analytical instruments could be inserted through either the fluid inlet or fluid outlet pipe (4, 5), however insertion and location of the instrument should be carried out in such a manner so as to minimise disturbance to fluid flow within the scaffold.

Referring to FIG. 3, in one embodiment of the invention, the tissue testing chamber (3), comprises a heart valve module (30). The heart valve module comprises a 3-dimensional scaffold of heart valve tissue (31) and is attached to the fluid inlet and outlet pipes (4, 5) by sutures or with a barbed fixture, thereby providing a channel for fluid flow between the two pipes (4, 5). In use, fluid enters the bioreactor (1) through the fluid inlet pipe (4) and into the scaffold of heart valve tissue (31).

Referring to FIG. 4 in a further embodiment of the invention the tissue testing chamber (3) comprises a vascular graft module (40). The vascular graft module (40) comprises a plurality of 3-dimensional scaffolds of vascular graft tissue (41). Vascular graft modules are especially suitable for testing arterial tissue. The vascular graft scaffolds (41) are attached to the fluid inlet and outlet pipes (4, 5) by sutures or with a barbed fixture. In use, fluid enters the bioreactor (1) through the fluid inlet pipe (4) and into the scaffolds of vascular graft tissue (41). It will be appreciated that having a plurality of scaffolds is more cost effective than having one scaffold as each scaffold can comprise a different type of tissue, therefore multiple tissue testing can be carried out at a relatively low cost.

Referring to FIG. 5, in a further embodiment of the invention, the bioreactor can further comprise a gel module (50). The gel module (50) is divided into a plurality of compartments (51) where each compartment comprises a gel matrix. Tissue can be grown in each of the compartments. In the case of this embodiment, fluid enters the tissue testing chamber (3) via the fluid inlet pipe (4). The fluid is then passed through each of the compartments (51) where it comes into contact with the tissue in the gel matrices. This type of construction of the bioreactor is particularly suitable for preliminary testing of the effects of different materials on tissue, and is also suitable for monitoring the uptake of stem cells by the tissue in the gel matrices.

Referring to FIG. 6, in a still further embodiment of the invention the bioreactor comprises a point bending module (60). The point bending module (60) is divided into a plurality of compartments (61), where each compartment can hold a different type of material. Within each compartment (61), there comprises an activating arm (not shown) which when pulsed can flex the tissue within each compartment (61) and is therefore suitable for preliminary testing of the physical characteristics of the tissue. In addition an electric current could be applied to the tissue for the evaluation of cardiac muscle tissue, and in the generation of cardiac patches.

Referring to FIG. 7 there is provided a process outline of a method of evaluating biological material. In step 101, a 3-dimensional scaffold of tubular biological material which replicates native tissue in vivo is formed. In one embodiment of the invention in step 102 labelled biomolecules are delivered to the scaffold. In step 103, the 3-dimensional scaffold of steps 101 and 102 are transferred to an environment which simulates physiological conditions.

In step 104 fluid is delivered through the scaffolds.

In step 105, the effect of a biomolecule on the biological material is evaluated by passing fluid comprising labelled biomolecules through the scaffold of step 101.

In step 106, the effect of implanting a medical device in the biological material is evaluated by implanting a medical device into the scaffold of either step 101 or 102.

In an alternative embodiment of the invention, in step 107 the medical device is coated with labelled biomolecules and is then implanted into the scaffold of step 101.

The scaffolds of steps 104, 105, 106 and 107 are analysed in step 108. The interaction between the test material and the biological material can be ascertained by visualising changes in shape and size of the cells within the biological material. Additionally gene expression techniques such as Polymerase Chain Reaction (PCR) can be used.

There are many different ways of analysing the biological material. If the biological material is being tested to evaluate the effect of a biomolecule, the biomolecule will have been labelled, either by magnetic labelling, radiolabelling or fluorescent labelling. The presence of the biomolecule can then be sensed using probes, cameras or sensors such as laser sensors. If the biological material is being tested to evaluate the effect of implanting a medical device into the biological material, the biological material can be monitored using a camera. Tearing or puncturing of the biological material can therefore be visualised. Testing of the fluid exiting the bioreactor also indicates whether the biomolecules adhered to or were absorbed by the biological material.

It will be appreciated that in each of the above embodiments that some or all of the analytical instruments can be connected to a PC. A computer program in combination with the analytical instruments can be used to both monitor and determine certain properties of the biological material. In addition to this, analytical formulae and computer based calculation methods can also assist in determining properties of the biological material.

For example, analysis of the external radial deformation of the biological material to varying pressure can be carried out using a camera. The pressure of the flow in the biological material can be measured using a flow sensor. Both instruments can be networked to a PC and changes in the radial deformation with varying pressure can be recorded using a PC and computer program.

It is also possible to determine the material, physical and/or biological properties of the biological material prior to carrying out any testing. Using the following analytical formulae (equations 1, 2 and 5) and computer based calculation methods (the finite element method and spreadsheet calculation methods) and based on the radial dimensions of the biological material, the pressure and the measured external radial deformation, the overall and/or effective elastic modulus of the biological material, the internal radial deformation and the hoop strain experienced by the inner lining of the biological material (such as the inner lining of the endothelial cells) can be determined.
P=internal pressure
E=overall/effective elastic/Young's modulus of the construct
v=Poisson's ratio of scaffold
E₁=elastic/Young's modulus of the collagen scaffold
E₂=elastic/Young's modulus of the collagen scaffold and smooth muscle cells
a=internal radius of construct
c=external radius of construct
b=intermediate radius of bi-layer smooth muscle cell/collagen construct (radius of proliferation of smooth muscle cells from internal surface)
uₐ=radial deformation at radius a (internal radial deformation)
u_{b}=radial deformation at radius b
u_{c}=radial deformation at radius c (external radial deformation)

With P, c, a and u_{c} known, E can be estimated from eqn. (1):

$$E = \frac{2 {Pca}^{2}}{{u}_{c} ( {c}^{2} - {a}^{2} )} \qquad ( 1 )$$

and uₐ can be estimated from eqn. (2):

$${u}_{a} = \frac{{Pa}^{3}}{E ( {c}^{2} - {a}^{2} )} [ ( 1 - v ) + ( 1 + v ) \frac{{c}^{2}}{{a}^{2}} ] \qquad ( 2 )$$

Additionally, based on the pressure, the elastic properties and the external radial deformation, the thickness of the smooth muscle cell layer can be determined using analytical formulae (equations 3, 4 and 5) and computer based calculation methods (the finite element method and spreadsheet calculation methods). In this way, smooth muscle cell proliferation can be quantified.

With P, c, a, u_{c}, E₁ and E₂ known, b can be estimated from eqn. (3) (by iteration using spreadsheet calculation software):

$${b}^{2} = {a}^{2} + \frac{\frac{4 {Pca}^{2}}{{u}_{c}} - {E}_{1} ( {c}^{2} - {b}^{2} ) [ ( 1 - v ) + ( 1 + v ) \frac{{a}^{2}}{{b}^{2}} ]}{{E}_{2} [ ( 1 - v ) + ( 1 + v ) \frac{{c}^{2}}{{b}^{2}} ]} \qquad ( 3 )$$

and uₐ can be estimated from eqn. (4):

$${u}_{a} = \frac{{Pa}^{3}}{{E}_{2} ( {b}^{2} - {a}^{2} )} [ ( 1 - v ) + ( 1 + v ) \frac{{b}^{2}}{{a}^{2}} ] - \frac{{u}_{c} {E}_{1} a ( {c}^{2} - {b}^{2} )}{{E}_{2} c ( {b}^{2} - {a}^{2} )} \qquad ( 4 )$$

The internal engineering hoop strain, eₗ, and the internal true hoop strain, εₜ, can be estimated from the following:

$${e}_{1} = \frac{{u}_{a}}{a} \\ {ɛ}_{t} = ln ( 1 + \frac{{u}_{a}}{a} ) \qquad ( 5 )$$

Subsequent to testing, the methods outlined above can also be used to determine the internal layer strain and the smooth muscle layer proliferation respectively. Thus an accurate evaluation of the biological material prior to and post testing can be performed.

Another example is if a test material such as a stent is deployed in the biological material, computer based calculation methods (the finite element method) can be used to determine the radial dimensions of the deformed construct.

### EXAMPLE 1

### Measurement of Mechanical Properties of Vascular Graft Tissue in the Bioreactor

A scaffold of vascular graft tissue was prepared as outlined previously and transferred to the bioreactor. The scaffold was sutured to the fluid inlet and outlet pipes. A camera was inserted into the interior of the scaffold. Fluid was delivered through the fluid inlet pipe in a pulsatile manner to provide an intraluminal pressure to the graft tissue. Magnified digital images of the interior of the graft tissue were obtained using the camera and the maximum and minimum distention of the graft were measured using the following equation:

$$\frac{Δ L}{L o}$$

$$where Δ L = change in length$$

$$L o = original length$$

$$or$$

$$\frac{Δ D}{D o}$$

$$where Δ D = change in diameter$$

$$D o = original diameter$$

Pressure transducers were also placed in the fluid inlet and outlet pipes and the pressure transducer measured the pressure required to burst the vascular graft tissue. A flow probe was also inserted into the scaffold via one of the fluid pipes and the flow rate was measured overtime.

### EXAMPLE 2

### Measurement of Mechanical Properties of Heart Valve Tissue in the Bioreactor

A scaffold of heart valve tissue was prepared as outlined previously and transferred to the bioreactor. The scaffold was sutured to the fluid inlet and outlet pipes.

Pressure transducers were also placed in the fluid inlet pipe and the fluid outlet pipe. The pressure transducers were used to measure the pressure of the fluid entering the valve tissue and exiting the valve tissue to record pressure changes over time. A pressure change was expected and indicated that the valve opened and closed. This is due to the fact that a valve causes a back pressure and thus a change in pressure.

A flow probe was also inserted into the scaffold via one of the fluid pipes and the flow rate was measured over time.

### EXAMPLE 3

### Analysis of the Anatomy of Vascular Graft Tissue in the Bioreactor

A scaffold of tissue engineered vascular graft was prepared as outlined previously stained with a fluorescent stain and transferred to the bioreactor. The scaffold was sutured to the fluid inlet and outlet pipes. A laser was then used to detect changes in fluorescent intensity. Damaged cells will not fluoresce and hence the biological properties can be monitored.

### EXAMPLE 4

### Analysis of Drug Uptake by Vascular Graft Tissue in the Bioreactor

A scaffold of vascular graft tissue was prepared as outlined previously and transferred to the bioreactor. The scaffold was sutured to the fluid inlet and outlet pipes. Blood comprising a drug was delivered through the fluid inlet pipe into the scaffold. The drug was labelled with a fluorescent marker. As the blood exited the fluid outlet pipe it was sampled. An absence of the labelled drug in the blood indicated that the drug was absorbed by the vascular graft tissue. Analysis of the blood was carried out using spectroscopic methods.

### EXAMPLE 5

### Analysis of Gene/Protein Expression within Vascular Graft Tissue in the Bioreactor

A scaffold vascular graft tissue was prepared as outlined previously stained with a fluorescent antibody and transferred to the bioreactor. The scaffold was sutured to the fluid inlet and outlet pipes. The gene of interest was fused with the gene for green fluorescent protein (GFP). As the gene of interest was expressed and its protein synthesised the GFP was synthesised also. When the GFP cells were illuminated under near-ultraviolet light it caused them to fluoresce a bright green. It was therefore possible to see when and where the gene of interest was expressed in living tissue.

### EXAMPLE 6

### Analysis of Antibody Adhesion to Vascular Graft Tissue in the Bioreactor

A scaffold of vascular graft tissue was prepared as outlined previously, stained with fluorescent antibodies and transferred to the bioreactor. The scaffold was sutured to the fluid inlet and outlet pipes. The fluorescent antibodies had been prepared by covalently binding the antibodies to the fluorescent dye fluorescein. Media was delivered through the fluid entry pipe into the scaffold. As the media exited through the fluid outlet pipe it was sampled and examined using a fluorescence activated cell sorter (FACS) to analyse whether the antibodies adhered to the vascular graft tissue or were removed with the media.

### EXAMPLE 7

### In Vivo Analysis of Biological Processes

A scaffold of vascular graft tissue was prepared as outlined previously and transferred to the bioreactor. The scaffold was sutured to the fluid inlet and outlet pipes. Positron emitting radiotracers were injected directly into the graft. The distribution path of the radiotracers was analysed using Positron Emission Topography and MicroPET.

Depending on the type of radiotracer used, the physiological, biochemical and pharmacokinetic properties of the graft were analysed. For example, the radiotracer technetium-99 labelled HM-PAO was used to measure blood flow.

### EXAMPLE 8

### Analysis of Temperature Change on Biological Material

A scaffold vascular graft tissue was prepared as outlined previously and transferred to the bioreactor. The scaffold was sutured to the fluid inlet and outlet pipes. The temperature in the tissue testing chamber was modified by the temperature controlled incubator. The effect of the temperature change on the tissue was analysed using Forward Looking Infra Red (FLIR) which is a thermal imaging apparatus. It was possible to differentiate living cells from dead cells due to the difference in temperature between them.

### EXAMPLE 9

### Analysis of the Effect of Implantation of a Medical Device into a Vascular Graft

A scaffold of vascular graft tissue was prepared as outlined previously and transferred to the bioreactor. The scaffold was sutured to the fluid inlet and outlet pipes. A stent was deployed using a balloon catheter through the fluid inlet pipe and was implanted into the vascular graft tissue. A camera was also inserted into the scaffold through one of the fluid pipes and the effect of the stent on the tissue was monitored.

### EXAMPLE 10

### Simultaneous Measurement of Mechanical Properties of a Medical Device and Physical Response and Biological Response of Vascular Graft Tissue

A scaffold of vascular graft tissue was prepared as outlined previously and transferred to the bioreactor. The scaffold was sutured to the fluid inlet and outlet pipes. A stent was obtained and was coated with a pharmaceutical drug. The stent was deployed using a balloon catheter through the fluid inlet pipe and was implanted into the vascular graft, where it expanded. A camera was also inserted into the scaffold through one of the fluid pipes and the effect of the stent on the tissue was monitored. Additionally the fluid exiting the fluid outlet pipe was sampled using spectroscopy to test for drug elution. An absence of the drug in the fluid indicated an adherence of the drug to the vascular graft tissue.

### EXAMPLE 11

### Simultaneous Measurement of Mechanical Properties of a Medical Device, and the Physical and Biological Response of Heart Valve Tissue

A scaffold of heart valve tissue was prepared as outlined previously and transferred to the bioreactor. The scaffold was sutured to the fluid inlet and outlet pipes. An artificial heart valve coated with a labelled drug was implanted into the tissue, and the effect was monitored both by using a camera and testing the fluid exiting the chamber.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiment hereinbefore described, but may be varied in both construction and detail.

1. A method of evaluating biological material comprising:
forming a 3-dimensional scaffold of tubular biological material;
delivering a fluid through the tubular biological material;
and evaluating the biological material;
characterised in that; the method further comprises the steps, not necessarily sequentially, of:
forming the 3-dimensional scaffold of tubular biological material so as to replicate native tissue in vivo;
transferring the biological material to an environment which simulates physiological conditions;
applying a test material to the biological material; and
analysing the interaction between the test material and the biological material.

2. A method as claimed in claim 1, wherein the test material comprises biomolecules and wherein the method further comprises:
labelling the biomolecules; and
delivering an amount of the labelled biomolecules to the biological material prior to transferring the biological material to the environment which simulates physiological conditions.

3. A method of evaluating biological material as claimed in claim 1 wherein the test material comprises biomolecules and wherein the method further comprises:
labelling the biomolecules;
adding an amount of the labelled biomolecules to the fluid;
delivering the fluid and labelled biomolecules through the tubular biological material;
allowing the fluid and labelled biomolecules to interact with the biological material;
analysing the interaction between the labelled biomolecules and the biological material.

4. A method of evaluating biological material as claimed in claim 1 wherein the test material comprises a medical device and wherein the method further comprises:
implanting the medical device into the tubular biological material;
allowing the medical device and the biological material to interact;
analysing the interaction between the medical device and the biological material.

5. A method as claimed in claim 4 further comprising the steps of:
labelling biomolecules;
coating the medical device with an amount of the labelled biomolecules prior to implanting the medical device into the biological material; and
analysing the interaction between the labelled biomolecules, medical device and biological material.

6. A method as claimed in claims 4 or 5 wherein the medical device is selected from a group consisting of one or more of stent, artificial heart valve, cardiac patch and vascular graft.

7. A method as claimed in claim 1, wherein the fluid is delivered at a rate of between 1 and 5 l/min.

8. A method as in claim 1, wherein the fluid is selected from a group consisting of one or more of physiological saline, aldehyde solution, isotonic saline solution, albumin solution or suspension, tissue culture medium and blood.

9. A method as claimed in claim 1, wherein the biomolecules are labelled using one or more of magnetic labelling, radiolabelling, fluorescent labelling and thermal imaging.

10. A method as claimed in claim 1, wherein the biological material is analysed using an instrument selected from the group consisting of one or more of probe, camera, sensor, laser and pressure transducer.

11. A method as claimed in claim 1, wherein the biological material is cardiovascular tissue selected from a group consisting of one or more of vascular graft tissue, heart valve tissue, artery tissue and cardiac muscle tissue.

12. A method as claimed in claim 1, wherein:
prior to applying the test material to the biological material, carrying out the further step of:
determining the material, physical, and/or biological properties of the biological material.

13. A method as in claim 1, wherein:
the biological material is transferred to a bioreactor which simulates physiological conditions; and the biological material is evaluated as a 3-dimensional scaffold under simulated physiological conditions within the bioreactor.

14. A bioreactor suitable for evaluating biological material according to the method as claimed in claim 1.

15. A computer program comprising program instructions for causing a computer to control the step of delivering the fluid through the tubular biological material according to the method as claimed in claim 1.

16. A computer program comprising program instructions for causing a computer to carry out the step of analysing the interaction between the test material and the biological material according to the method as claimed in claim 1.

17.-20. (canceled)
